(19) United States
(12) Patent Application Publication
(10) Pub. No.: US 2003/0215868 A1
(43) Pub. Date: Nov. 20, 2003

(54) Method of detecting schizophrenia risk

(76) Inventors: Philip Seeman, Toronto (CA); Gabriela Novak, Mississauga (CA); Teresa Tallerico, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/437,931

(22) Filed: May 15, 2003

(30) Foreign Application Priority Data
May 16, 2002 (GB) 0211212.6

(51) Int. Cl. C12Q 1/68; C12P 19/34

(52) U.S. Cl. 435/6; 435/91.2

(57) ABSTRACT

Methods and kits for determining susceptibility of a patient to neuropsychiatric disorders are described. The method involves analyzing a sample comprising nucleic acids from a patient for a polymorphism of the Nogo gene.

The present application claims priority from United Kingdom patent application number 0211212.6, filed on May 16, 2002.

### FIELD OF THE INVENTION

The present invention relates to methods and kits for detecting neuropsychiatric disorders such as schizophrenia.

### BACKGROUND OF THE INVENTION

Although schizophrenia is a heritable disorder, the genes responsible for this disease remain to be identified (33,34). The inventors hypothesize that schizophrenia may arise from abnormal neurodevelopment due to aberrant neuron formation and migration, or altered expression of proteins needed for brain development (3,7). One such critical protein may be Nogo (also known as reticulon 4, RTN4, NI 250 or RTN-X), which has been shown to be highly expressed by oligodendrocytes in CNS myelinated tissues during rat fetal development and in the adult rat (19,21). Myelin develops in vivo at about the same time as the CNS loses plasticity, and myelination appears to be synchronized with the gradual loss of neuronal regenerative ability (31). Nogo inhibits the outgrowth of neurites and nerve terminals (9,15,29), and may have an important role in regulating neuronal migration and plasticity. Nogo is hypothesized to constitutively suppress in vivo gene expression of neuronal transcription factors, e.g., c-Jun and JunD, which are associated with neuronal growth (36), and has been shown to inhibit neuronal sprouting of injured and uninjured Purkinjie neurons in the adult rat cerebellum (6). Nogo is also expressed by neurons during development, however, there is no known functional role for neuronal Nogo (19,21). The Nogo gene encodes for three alternatively spliced variants, Nogo-A, Nogo-B, and Nogo-C, which share a common C-terminal domain of 188 amino acids containing two putative transmembrane domains, and an endoplasmic reticulum retention motif (31). It is not yet clear where the neurite inhibitory domain resides. Two studies have suggested a Nogo-A-specific region (9,29), while a third demonstrated inhibitory activity in a domain common to all three isoforms (15). Nogo, therefore, functions as a developmentally regulated (19,21) tonic inhibitor of neuronal growth through negative, constitutive down-regulation of growth-associated genes (5,18,36). Since Nogo has an important role in synaptic plasticity and neuronal migration, the altered expression of this gene in schizophrenia may contribute to the abnormal neuronal organization observed in schizophrenia.

There is a need in the art to develop new methods for detecting schizophrenia.

### SUMMARY OF THE INVENTION

To search for genes with altered mRNA expression in schizophrenia brain, the inventors carried out cDNA library subtractive hybridization experiments using RNA extracted from schizophrenia and control post-mortem frontal cortices (12). The brain region selected for study was the prefrontal cortex, important for memory and cognition. These faculties are often impaired in schizophrenia (14), suggesting that there may be changes in gene expression and/or neuronal organization. These studies identified Nogo as being potentially over-expressed in schizophrenia. The gene expression of Nogo was therefore measured in seven schizophrenia and seven control frontal cortex samples by quantitative reverse transcription-polymerase chain reaction (17). To identify possible polymorphisms in this gene, the Nogo nucleotide sequence was determined in a series of schizophrenia and control samples. The Nogo mRNA contains a CAA insert polymorphism in the 3′-untranslated region, 669 bases beyond the coding region. The prevalence of individuals homozygous for the CAA insert was significantly higher in schizophrenia compared to controls.

The present invention therefore provides a method of determining the susceptibility of a patient to a neuropsychiatric disorder comprising:

(a) obtaining a sample from a patient; and

(b) testing the sample for the presence of a polymorphism in the Nogo gene, wherein the presence of polymorphism indicates that the patient is susceptible to a neuropsychiatric disorder.

The polymorphism is preferably a CAA insert at nucleotides 4386-4388 of the Nogo sequence shown in FIG. 1 (SEQ ID NO:1). In a specific embodiment, the patient is homozygous for the CAA polymorphism and the patient has schizophrenia.

The present invention further relates to methods of diagnostic evaluation, genetic testing and prognosis for a neuropsychiatric disorder in a patient.

The present invention also provides a kit for determining susceptibility of a patient to a neuropsychiatric disorder, for diagnosing a neuropsychiatric disorder or for determining if a patient will have increased symptomatology associated with a neuropsychiatric disorder, comprising reagents necessary for determining the presence of a polymorphism in the directions for its use.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows the partial Nogo cDNA sequence. The CAA polymorphism in the 3′-untranslated region is located at nucleotides 4386-4388 and is often associated with a TATC sequence deletion at nucleotide 3905. The TGA is the stop codon for the coding sequence.

### DETAILED DESCRIPTION OF THE INVENTION

To search for genes overexpressed in schizophrenia, cDNA library subtractive hybridization experiments between post-mortem human frontal cerebral cortices from schizophrenia individuals and neurological controls were carried out. One of the genes over-expressed in schizophrenia was identified as Nogo (also known as reticulon 4, RTN4, NI 250, or RTN-X), a myelin-associated protein which inhibits the outgrowth of neurites and nerve terminals. The elevated expression of Nogo mRNA in schizophrenia has been confirmed by quantitative reverse transcription-polymerase chain reaction studies, 16.84 pg Nogo cDNA/μg total RNA in schizophrenia and 10.42 pg Nogo cDNA/μg total RNA in controls (n=7; p=0.01, T-test for n<30). To identify possible polymorphisms in this gene, the Nogo nucleotide sequence was determined in a series of schizophrenia and control samples. The Nogo mRNA contains a CAA insert polymorphism in the 3′-untranslated region. The prevalence of individuals homozygous for the CAA insert was significantly higher in schizophrenia compared to controls in genomic DNA samples extracted from post-mortem brain and blood samples, 17/81 or 21% in schizophrenia and 2/61 or 3% in controls (p=0.0022, Chi-square and Fisher's Exact Tests). The 3′-untranslated regions of eukaryotic genes have been shown to regulate gene expression. The increased frequency of the Nogo CAA insert polymorphism in schizophrenia may contribute to abnormal regulation of Nogo gene expression, and may suggest a role for Nogo in the disturbed neurodevelopment observed in schizophrenia.

The present invention therefore provides a method of determining the susceptibility of a patient to a neuropsychiatric disorder comprising:

(a) obtaining a sample from a patient; and

(b) testing the sample for the presence of a polymorphism in the Nogo gene, wherein the presence of a polymorphism indicates that the patient is susceptible to a neuropsychiatric disorder.

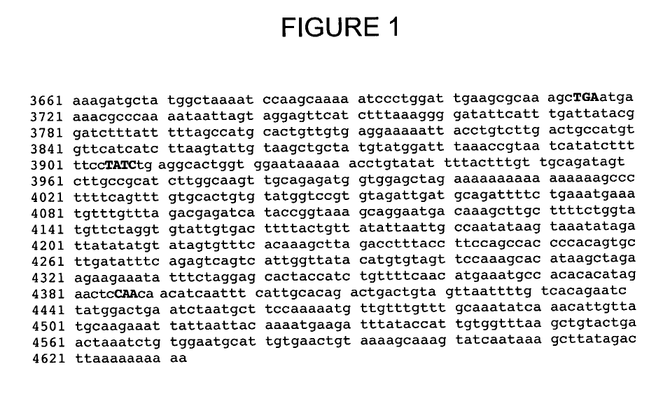

In one embodiment, the polymorphism is a CAA insert at nucleotides 4386-4388 of the Nogo sequence shown in FIG. 1 (SEQ ID NO:1). Accordingly, the present invention therefore provides a method of determining the susceptibility of a patient to a neuropsychiatric disorder comprising:

(a) obtaining a sample from a patient; and

(b) testing the sample for the presence of a polymorphism in the Nogo gene, wherein the polymorphism is the presence of a CAA insert at nucleotides 4386-4388 of the Nogo sequence shown in FIG. 1 (SEQ ID NO:1), and the presence of the polymorphism indicates that the patient is susceptible to a neuropsychiatric disorder.

The “Nogo gene” as used herein is a gene that is over-expressed in patients with schizophrenia and is also known as reticulon 4, RTN4, NI250 or RTN-X. The term “Nogo gene” includes any and all of the splice variants of Nogo such as Nogo-A, Nogo-B and Nogo-C. The complete sequence of the Nogo-A (which includes the sequence of Nogo-B and the terminal portion of Nogo-C) is deposited in GenBank under accession number AF148537. A partial cDNA sequence of Nogo showing the CAA polymorphism is shown in FIG. 1 (SEQ ID NO:1).

The term “neuropsychiatric disorder” refers to any type of neuropsychiatric disorder including, but not limited to, schizophrenia, bipolar disorder, unipolar disorder, dsythmic disorder, depression, seasonal affective disorder.

The term “schizophrenia” refers to an illness of young adults with typical clinical symptoms of delusions, hallucinations, paranoia, loss of contact with reality, social withdrawal, poverty of speech, with possible depression or mania, and requiring antipsychotic medication without which the illness rarely improves. The risk of suicide is high in an untreated individual with schizophrenia or psychosis.

In a specific embodiment, the patient is homozygous for the CAA insert and suffers from schizophrenia. The inventors have also determined that many schizophrenic patients with the CAA polymorphism also have a TATC deletion at nucleotide 3905. Accordingly, the present invention provides a method of determining the susceptibility of a patient to a neuropsychiatric disorder comprising:

(c) obtaining a sample from a patient; and

(d) testing the sample for the presence of a CAA insert at nucleotides 4386-4388 and a TATC deletion at nucleotide 3905 of the Nogo sequence shown in FIG. 1 (SEQ ID NO:1), wherein the presence of the CAA insert and the TATC deletion indicates that the patient is susceptible to a neuropsychiatric disorder.

The sample obtained from the patient can be any biological sample containing nucleic acids including, but not limited to, blood, urine, skin, hair, sperm, buccal mucosa as well as tissue samples and fractions of any of the foregoing.

The sample may be tested for the presence of a polymorphism in the Nogo gene (such as the CAA insert) using a variety of techniques known in the art. Generally, nucleic acids comprising the 3′ untranslated region of the Nogo gene, or a portion thereof, are obtained from the sample and amplified using the Polymerase Chain Reaction (PCR) using primers to that region. Examples of specific primers are described in Example 1 and SEQ ID NOS:2-6. By “a portion thereof” it is meant a sufficient portion of the Nogo 3′ untranslated region to allow the identification of a polymorphism, in particular the CAA insert at nucleotides 4386-4388 of the Nogo sequence shown in FIG. 1 (SEQ ID NO:1). The PCR products can be subjected to any method that would allow one to identify the presence of a polymorphism. In one embodiment, the PCR products may be subjected to an electrophoretic assay (such as gel electrophoresis or capillary electrophoresis) to determine the relative size of the PCR product. For example, the size of the PCR product can be determined by comparing its migration on an electrophoresis gel with a 200 bp ladder. Once the size has been determined in this manner, it can be compared with the predicted size of the Nogo alleles to confirm its identity.

In another embodiment, the PCR products may be probed with a fluorescently-labeled nucleic acid sequence specific for a region in the 3′ untranslated region. In a further embodiment, the PCR products may be sequenced using techniques known in the art including commercially available sequencing kits to determine if a polymorphism is present in the sample. Other sequencing technologies such as Denaturing High Pressure Liquid Chromatography or mass spectroscopy may also be employed.

In yet another embodiment, detection of a polymorphism can be performed by using restriction enzymes or Single Stranded Conformation Polymorphism (SSCP) techniques. In addition, methods for high throughput detection of nucleotide polymorphisms using allele-specific probes may be used such as DNA chip technology. The design and use of allele-specific probes for analyzing polymorphisms is described in, for example, Saiki et al., 1986. Saiki, 1989 and Dattagupta. Allele-specific probes can be designed that hybridize to a segment of target DNA from one patient but do not hybridize to the corresponding segment from another patient due to the presence of different polymorphic forms in the respective segments from the two patients. This technique may be used in high-through put or non-high-through put formats. Combinations of any of the above methods may be used.

As stated above, the present invention also relates to methods of diagnostic evaluation, genetic testing and prognosis for a neuropsychiatric disorder, in a patient. Accordingly, there is included in the present invention, a method of diagnosing a neuropsychiatric disorder in a patient by analyzing for the presence of a polymorphism in the Nogo gene in a biological sample obtained from the patient. In embodiments of the invention, the presence of a polymorphism in the Nogo gene, in particular, the CAA insert at bases 4386-4388 of the Nogo sequence shown in FIG. 1 (SEQ ID NO:1), indicates a likelihood that the patient is suffering from a neuropsychiatric disorder.

There is also included in the present invention, a method of determining if a patient will have increased symptomatology associated with a neuropsychiatric disorder, by analyzing for the presence of a polymorphism in the Nogo gene in a biological sample obtained from the patient. In embodiments of the invention, the presence of a polymorphism in the Nogo gene, in particular, the CAA insert at bases 4386-4388 of the Nogo sequence shown in FIG. 1 (SEQ ID NO:1), indicates a likelihood that the patient will have increased symptomatology associated with a neuropsychiatric disorder.

The method of the present invention may be used in combination with similar screens for other susceptibility markers for neuropsychiatric disorders.

The invention also includes kits for use in the above methods for detecting the presence of a polymorphism in the Nogo gene. Accordingly, the present invention provides a kit for determining the susceptibility of a patient to a neuropsychiatric disorder, for diagnosing neuropsychiatric disorders or for determining if a patient will have increased symptomatology associated with a neuropsychiatric disorder, comprising reagents necessary for determining the presence of a polymorphism in the Nogo gene and directions for its use. In one embodiment, the kit is for determining the susceptibility of a patient to schizophrenia comprising reagents necessary for determining the presence of a CAA insert at bases 4386-4388 of the Nogo sequence shown in FIG. 1 (SEQ ID NO:1) and directions for its use.

The reagents useful in the kit can be determined by one of skill in the art and can include primers to the appropriate regions of the Nogo gene in order to amplify nucleic acids from a test sample using PCR. The kit may further include nucleic acid probes useful in determining the presence of a polymorphism in the Nogo gene. The kit may also include electrophoretic markers such as a 200 bp ladder. Other components of the kit can include nucleotides, enzymes and buffers useful in a method of the invention. As an example, a kit of the invention may include primers for amplifying the region surrounding the promoter region, DNA polymerase, each of dATP, dTTP, dCTP and dGTP, 7-deaza-dGTP, 10 mM Tris-HCl, 50 mM KCl, 1.5 mM MgCl₂ and 5% DMSO. The kit will also include detailed instructions for carrying out the method for detecting the presence of a polymorphism in the Nogo gene.

The following non-limiting examples are illustrative of the present invention:

### EXAMPLE

Materials and Methods

Post-mortem tissues: Post-mortem human frontal cortical brain tissues (BA10) were provided by the Stanley Foundation Neuropathology Consortium (Bethesda, Md.) from a collection of 60 brains, fifteen each from individuals diagnosed (DSMIV criteria) with either schizophrenia, bipolar disorder, non-psychotic depression or neurologically normal (Tables 2 and 3) (32). Post-mortem human brain frontal cortices were also dissected by, and obtained from, the Canadian Brain Tissue Bank (Toronto, ON, Canada) and the National Neurological Research Specimen Bank (West Los Angeles VA Medical Center, Los Angeles, Calif.). The diagnosis of schizophrenia was made by two psychiatrists independently examining the case records and using DSM-IV criteria (American Psychiatric Association, 1987) (1).

Blood samples: Additional control genomic DNA samples of 34 anonymous Caucasian individuals were obtained from relatives of rheumatic arthritis patients (laboratory of Dr. K. Siminovitch, Mount Sinai Hospital, Toronto, ON). Genomic DNA was also obtained from blood samples from individuals diagnosed with schizophrenia from Dr. J. Lieberman (Hillside Hospital, NY; 15 samples), Dr. C. Shammi and Dr. C. Hudson (Centre for Addiction and Mental Health, Clarke site, Toronto, ON; 32 samples). Additional genomic DNA samples were obtained from Dr. B. O'Dowd (Toronto, ON; 72 samples) from individuals diagnosed with alcohol addiction (as well as substance abuse in some cases).

Quantitative RT-PCR: Total RNA was extracted from brain tissue samples using TRIzol Reagent (Life Technologies/Invitrogen) according to the manufacturer's guidelines.

Complementary DNA was prepared from 2 μg of total RNA using the SuperscriptTM Preamplification System for First Strand cDNA Synthesis (Life Technologies/Invitrogen). An internally truncated standard for competitive PCR experiments was prepared with a 75 bp deletion (bases 4460-4534, AF148537) using a reverse primer with sequences flanking the desired deletion (5′-TACAGCTTAAACCACAATGGTAGCATTAGATTCAGTCC-3′ (SEQ ID NO:2), complementary to bases 4556-4535, 4459-4444) and a forward primer (5′-GCATCAGGCACAGATAGATC-3′ (SEQ ID NO:3), bases 3612-3630). The truncated Nogo PCR product was amplified using Qiagen Taq™ Polymerase, purified using QIAEX II Gel Extraction kit (Qiagen Inc., Mississauga, Ontario), and quantitated by A260 UV absorbance. The competitor template had identical primer annealing sites and similar base composition as the Nogo cDNA under study, resulting in accurate estimates of transcript levels. The primers used for PCR amplification were the above forward primer (bases 3612-3630) and a reverse primer (5′-TACAGCTTAAACCACAATGGTA-3′ (SEQ ID NO:4), complementary to bases 4556-4535), yielding predicted PCR products of 945 bp from Nogo cDNA and 870 bp from the competitor template. The primers were designed to amplify all three splice variants of Nogo: Nogo-A, Nogo-B, and Nogo-C. To quantitate Nogo gene expression in frontal cortex cDNA, several dilutions of the competitor template were co-amplified in PCR reactions with a constant amount of cDNA. The amplified DNA was size-fractionated by electrophoresis on agarose gels containing ethidium bromide. The amount of stained DNA amplified (endogenous Nogo and competitor) from each sample were quantified using the Bio-Rad Gel Doc Imaging System. The amount of endogenous Nogo cDNA in each reaction was determined by comparison with the level of the coamplified Nogo competitor DNA. That is, the ratios for each dilution of competitor template input and endogenous Nogo was compared to determine the point of equivalence.

Genomic DNA extraction: Total DNA was extracted from brain tissue samples using TRIzol Reagent (Life Technologies/Invitrogen) according to the manufacturer's guidelines. Genomic DNA was extracted from blood samples by standard methods. Briefly, 3 ml of blood were mixed with 9 ml of a solution containing 155 mM NH₄Cl, 10 mM KHCO3 and 0.1 mM EDTA. After centrifugation at 3000×g for 10 min at 4° C., the remaining tissue was digested for 3 h at 55° C. with proteinase K (125 μg/ml) in 3 ml of nucleus-lysing buffer (75 mM NaCl, 25 mM EDTA, 1% SDS). After centrifugation (3,000×g for 10 min), the DNA was precipitated with ethanol, dried, and dissolved in 500 μl of buffer (10 mM Tris, 1 mM EDTA; pH 8) and stored at 4° C.

Amplification and sequencing of the Nogo DNA template: Genomic DNA was PCR amplified using the Qiagen Taq™ Polymerase Kit (Qiagen Inc., Mississauga, Ontario). Primers used for amplification of the Nogo gene, AF148537, were forward primer (5′-TTACCTGTCTTGACTGCC-3′ (SEQ ID NO:5), bases 3819-3836) and reverse primer (5′-TACAGCTTAAACCACAATGG-3′ (SEQ ID NO:6), complementary to bases 4556-4537). PCR products were analyzed using agarose gel electrophoresis before sequence analysis using Sequenase Version 2.0 PCR Product Sequencing Kit (product # 70170, USB) and the above described reverse primer.

Results and Discussion

An internally deleted standard was prepared and used to quantitate the total amount of Nogo transcript (Nogo-A, Nogo-B, and Nogo-C) in each sample. The clinical summaries and Nogo gene expression are presented in Table 1. The mean expression in controls was 10.42 pg Nogo cDNA/μg total RNA and in schizophrenia was 16.84 pg Nogo cDNA/μg total RNA, a 1.6-fold increase in disease (p=0.01, T-test for n<30). These data were presented at the 31st Annual Meeting of the Society for Neuroscience (San Diego, Calif., USA, 2001).

Nucleotide sequence analyses of schizophrenia and control DNA samples revealed a polymorphism in the Nogo cDNA, a single CAA insert in the 3′-untranslated region (bases 4386-4388, Genbank AF148537), as indicated in FIG. 1. The frequency of the CAA insert polymorphism was examined in neurological controls and in samples from individuals who had schizophrenia. The prevalence of individuals homozygous for the CAA insert in the schizophrenia samples was 17 out of 81 or 21%, in contrast to 2 out of 61 or 3% in the control samples (Table 2). This difference was found to be statistically highly significant, using the Chi-square and Fisher's Exact Tests (p=0.0022 for both tests). If only genomic DNA extracted from post-mortem brain samples is considered, the difference increases to 38% in schizophrenia (n=32) versus 0% in controls (n=27), and is highly significantly different (p=0.00058; Fisher's Exact Test for small samples).

Almost all the DNA samples were from Caucasians. Of the twelve individuals homozygous for the CAA insert, two were Asian (one schizophrenia patient of Asian descent was not homozygous for the insert). The overall CAA allele frequency is 0.41 in schizophrenia and 0.22 in controls. In the well-matched Stanley brain bank samples, the CAA allele frequency is 0.5 in schizophrenia and 0.1 controls. This is in contrast to the blood samples, where the CAA allele frequency is 0.35 in schizophrenia and 0.25 in controls. These data suggest there are population differences for this Nogo polymorphism which will require a large set of well-matched samples to elucidate. The CAA insert is in most cases co-inherited with a TATC deletion at base 3905, 188 bases 3′ of the stop codon (FIG. 1). All individuals homozygous for the CAA insert are also homozygous for the TATC deletion, except for one schizophrenia sample and one control sample. Three individuals, one each from the bipolar, major depression, and schizophrenia samples were homozygous for the TATC deletion, but were not homozygous for the CAA insert. To search for additional polymorphisms, the complete Nogo cDNA sequence was determined for 9 individuals: four schizophrenia, one major depression, and four control samples. No additional common polymorphisms were identified, and the three polymorphisms listed for Nogo in the NCBI SNP database (C→T, 1343; C→G, 2850; C→A, 3751), were not present in the 9 samples completely sequenced. Furthermore, two of the three NCBI listed polymorphisms at positions 1343 and 3751 were not present in an additional independent set of 10 samples studied. The inventors did identify two additional polymorphisms, each observed in only one sample, a GTTT deletion (bases 4473-4476) and an ATT insertion at position 4508.

The inventors also observed that individuals with early-onset schizophrenia (before 20 years of age) were much less likely to carry the CAA insert, but due to the small sample size the inventors could not draw any conclusions. That is, the Stanley Brain Bank samples (n=15) revealed that none of the five schizophrenia individuals aged 13 to 19 years were homozygous for the CAA insert, while six of the ten schizophrenia individuals with disease onset at age 20 to 42 years were homozygous for the CAA insert.

The inventors also analyzed the prevalence of the CAA insert in bipolar and depressed individuals and in alcoholics (Table 3). It has been hypothesized that schizophrenia and affective disorders may share some genetic susceptibility (4,24). In our small study of bipolar and major depression samples, the inventors did identify 2/15 (15%) bipolar and 1/15 (7%) major depression samples homozygous for the CAA insert, however, the samples size is too small to comment on the possible role of the slightly increased prevalence of the CAA insert in affective disorders. Several individuals with alcohol addictions were homozygous for the CAA insert (11/72 or 15%). The schizophrenia genotype may, however, be over-represented in this group, because schizophrenia patients have a high prevalence of alcohol addiction [8]. Not surprisingly, the alcoholic group was statistically significantly different from the control group (p=0.02, Fisher's Exact Test).

In a group of African and Afro-American samples, the prevalence of the CAA insert was 3 out of 60, or 5%, in schizophrenia DNA samples, in contrast to 2 out of 111 or 1.8% in control DNA samples. However, while these frequency data were too low for statistical analysis, there was a 2.8-fold increased prevalence of the homozygous CAA insert polymorphism in schizophrenia.

The 5′- and 3′-untranslated regions (UTR) of eukaryotic mRNAs have been shown to regulate several aspects of gene expression. In particular, the 3′-UTR is involved in the regulation of mRNA stability (25), translation initiation (16), and mRNA subcellular localization (20). In neurons, several mRNAs have been demonstrated to be targeted to dendrites via 3′-UTR motifs (23,26). Mutations in the 3′-UTR are associated with a number of diseases, including neuroblastoma, myotonic dystrophy and a-thalassemia (10). The functional role of the CAA insert polymorphism in the Nogo 3′-UTR is unknown, and it does not match any known 3′-UTR functional motifs (28). However, polymorphisms in the Nogo 3′-UTR could contribute to altered expression of the Nogo gene in schizophrenia.

The gene for Nogo is located on chromosome 2p13-14 (27,35), and genome scans have found schizophrenia to be associated or linked to this region, 2p15-p12 (11,30). The present findings, therefore, offer new support to this chromosomal region as a site of schizophrenia susceptibility. Increased Nogo mRNA expression in schizophrenia as described above may lead to elevated levels of Nogo protein expression, and may therefore result in increased Nogo receptor stimulation. The Nogo receptor is located on chromosome 22q11 (13), a region strongly associated with schizophrenia (2,22). Nogo and its receptor may therefore be of considerable relevance to the biological basis of schizophrenia.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Quantitative RT-PCR of Nogo gene expression and clinical summaries of samples.

| Brain/ | P.M. | | Diagnosis, | Duration | Antipsychotic | pg Nogo cDNA per |
|---|---|---|---|---|---|---|
| Age/sex | delay(h) | Death | symptoms | (psychosis) | Rx | μg total RNA ± SE |
| Schizophrenia | | | | | | |
| tissues: | | | | | | |
| T1149/22/m | 18 | Suicide | Paranoid | 5 years | Flupenthixol | 9.68 ± 1.77 |
| T1176/19/m | 52 | Suicide | Depression | 1 year | None | 19.36 ± 4.33 |
| T1293/29/m | 4 | Drowning | Halluc., depr. | 13 years | Risperidone | 21.84 ± 7.06 |
| L477/82/f | 16 | Cardiac | Delusions | 36 years | Fluphenazine | 11.91 ± 2.82 |
| L695/30/m | 32 | Cardiac | Delusions | 9 years | Thioridazine | 20.35 ± 6.44 |
| L707/28/m | 41 | Suicide jump | Depression | 2 years | Off fluphenazine 4 yrs | 11.91 ± 3.62 |
| L1755/74/m | 14 | Cancer | Depression | 30 years | Trifluperazine | 20.35 ± 6.44 |
| Average | | | | | | 16.49 ± 1.83* |
| Control tissues: | | | | | | |
| T1214/92/m | 3 | Tachycardia | Control | None | | 4.22 ± 1.43 |
| T1225/31/m | 11 | Car accident | Control | None | | 19.36 ± 4.33 |
| T1367/80/m | 16 | Cancer | Control | None | | 2.82 ± 0.90 |
| L451/25/m | 14 | Drowning | Control | None | Phencyclidine abuser | 16.88 ± 1.98 |
| L475/77/f | 19 | Car accident | Control | None | | 7.94 ± 2.79 |
| L480/66/m | 19 | Cardiac infarct | Control | None | | 16.38 ± 6.09 |
| L1768/71/m | 25 | Cardiac; cancer | Control | None | | 4.07 ± 1.61 |
| Average | | | | | | 10.24 ± 1.69 |

Abbreviations:
L indicates brain tissue from the National Neurological Research Specimen Bank in Los Angeles; T indicates brain tissue from the Canadian Brain Tissue Bank in Toronto.
Standard error (SE) for samples based on standard deviation of each sample. SE for average based on standard deviation of the whole (control or schizophrenia) group.
*Statistically significantly different from controls (p = 0.01, T-test for n < 30).

TABLE 2
Frequency of CAA insert in schizophrenia and controls.

| | Total number | Homozygous | Heterozygous | Homozygous |
|---|---|---|---|---|
| | of samples | for CAA insert | for CAA insert | for no insert |
| Schizophrenia: | | | | |
| Stanley Bank | 15 | 6** | 3 | 6 |
| Other Banks | 17 | 6** | 6 | 5 |
| Blood DNA | 49 | 5 | 24 | 20 |
| Total | 81 | 17 (21% of 81)* | 33 | 31 |
| Controls: | | | | |
| Stanley Bank | 15 | 0 | 3 | 12 |
| Other Banks | 12 | 0 | 7 | 5 |
| Blood DNA | 34 | 2 | 13 | 19 |
| Total | 61 | 2 (3% of 61) | 23 | 36 |

*Significantly different from controls (p = 0.0022, Chi-square and Fisher's Exact Tests)
**Combined schizophrenia brain bank tissues significantly different from control brain bank tissues (p = 0.00058, Fisher's Exact Test)

TABLE 3
Frequency of CAA insert in disease and controls.

| | Total | Homozygous | Heterozygous | Homozygous |
|---|---|---|---|---|
| | samples | for CAA insert | for CAA insert | for no insert |
| Schizophrenia (Bethesda) | 15 | 6 (40% of 15) | 3 | 6 |
| Bipolar disorder (Bethesda) | 15 | 2 (13% of 15) | 4 | 9 |
| Depression (Bethesda) | 15 | 1 (7% of 15) | 9 | 5 |
| Control (Bethesda) | 15 | 0 | 3 | 12 |
| Alcoholics (Toronto) | 72 | 11 (15% of 72)* | 32 | 29 |

*Significantly different from controls (p = 0.02, Fisher's Exact Test)

### FULL CITATIONS FOR REFERENCES REFERRED TO IN THE SPECIFICATION

1. A.P. Association, Diagnostic and Statistical Manual of Mental Disorders., 4 edn., American Psychiatric Association, Washington, D.C., 1994.

2. A. S. Bassett, K. Hodgkinson, E. W. Chow, S. Correia, L. E. Scutt and R. Weksberg, 22q11 deletion syndrome in adults with schizophrenia, Am. J. Med. Genet. 81 (1998) 328-37.

3. F. M. Benes, Emerging principles of altered neural circuitry in schizophrenia, Brain Res. Brain Res. Rev. 31 (2000) 251-69.

4. W. H. Berrettini, Are schizophrenic and bipolar disorders related? A review of family and molecular studies, Biol. Psychiatry 48 (2000) 531-8.

5. P. A. Brittis and J. G. Flanagan, Nogo domains and a Nogo receptor: implications for axon regeneration, Neuron 30 (2001) 11-4.

6. A. Buffo, M. Zagrebelsky, A. B. Huber, A. Skerra, M. E. Schwab, P. Strata and F. Rossi, Application of neutralizing antibodies against NI-35/250 myelin-associated neurite growth inhibitory proteins to the adult rat cerebellum induces sprouting of uninjured purkinje cell axons, J. Neurosci. 20 (2000) 2275-86.

7. W. E. Bunney and B. G. Bunney, Evidence for a compromised dorsolateral prefrontal cortical parallel circuit in schizophrenia, Brain Res. Brain Res. Rev. 31 (2000) 138-46.

8. E. Cantor-Graae, L. G. Nordstrom and T. F. McNeil, Substance abuse in schizophrenia: a review of the literature and a study of correlates in Sweden, Schizophr. Res. 48 (2001) 69-82.

9. M. S. Chen, A. B. Huber, M. E. van der Haar, M. Frank, L. Schnell, A. A. Spillmann, F. Christ and M. E. Schwab, Nogo-A is a myelin-associated neurite outgrowth inhibitor and an antigen for monoclonal antibody IN-1, Nature 403 (2000) 434-9.

10. B. Conne, A. Stutz and J. D. Vassalli, The 3′ untranslated region of messenger RNA: A molecular ‘hotspot’ for pathology?, Nat. Med. 6 (2000) 637-41.

11. H. Coon, M. Myles-Worsley, J. Tiobech, M. Hoff, J. Rosenthal, P. Bennett, F. Reimherr, P. Wender, P. Dale, A. Polloi and W. Byerley, Evidence for a chromosome 2p13-14 schizophrenia susceptibility locus in families from Palau, Micronesia, Mol. Psychiatry 3 (1998) 521-7.

12. L. Diatchenko, Y. F. Lau, A. P. Campbell, A. Chenchik, F. Moqadam, B. Huang, S. Lukyanov, K. Lukyanov, N. Gurskaya, E. D. Sverdlov and P. D. Siebert, Suppression subtractive hybridization: a method for generating differentially regulated or tissue-specific cDNA probes and libraries, Proc. Natl. Acad. Sci. USA 93 (1996) 6025-30.

13. A. E. Fournier, T. GrandPre and S. M. Strittmatter, Identification of a receptor mediating Nogo-66 inhibition of axonal regeneration, Nature 409 (2001) 341-6.

14. P. S. Goldman-Rakic and L. D. Selemon, Functional and anatomical aspects of prefrontal pathology in schizophrenia, Schizophr. Bull. 23 (1997) 437-58.

15. T. GrandPre, F. Nakamura, T. Vartanian and S. M. Strittmatter, Identification of the Nogo inhibitor of axon regeneration as a Reticulon protein, Nature 403 (2000) 439-44.

16. N. K. Gray and M. Wickens, Control of translation initiation in animals, Annu. Rev. Cell. Dev. Biol. 14 (1998) 399-458.

17. G. Haberhausen, J. Pinsl, C. C. Kuhn and C. Markert-Hahn, Comparative study of different standardization concepts in quantitative competitive reverse transcription-PCR assays, J. Clin. Microbiol. 36 (1998) 628-33.

18. A. B. Huber and M. E. Schwab, Nogo-A, a potent inhibitor of neurite outgrowth and regeneration, Biol. Chem. 381 (2000) 407-19.

19. A. B. Huber, O. Weinmann, C. Brosamle, T. Oertle and M. E. Schwab, Patterns of nogo mRNA and protein expression in the developing and adult rat and after CNS lesions, J. Neurosci. 22 (2002) 3553-67.

20. R. P. Jansen, mRNA localization: message on the move, Nat. Rev. Mol. Cell. Biol. 2 (2001) 247-56.

21. A. Josephson, J. Widenfalk, H. W. Widmer, L. Olson and C. Spenger, NOGO mRNA expression in adult and fetal human and rat nervous tissue and in weight drop injury, Exp. Neurol. 169 (2001) 319-28.

22. M. Karayiorgou, M. A. Morris, B. Morrow, R. J. Shprintzen, R. Goldberg, J. Borrow, A. Gos, G. Nestadt, P. S. Wolyniec, V. K. Lasseter and et al., Schizophrenia susceptibility associated with interstitial deletions of chromosome 22q11, Proc. Natl. Acad. Sci. USA 92 (1995) 7612-6.

23. M. A. Kiebler and L. DesGroseillers, Molecular insights into mRNA transport and local translation in the mammalian nervous system, Neuron 25 (2000) 19-28.

24. W. Maier, D. Lichtermann, J. Minges, J. Hallmayer, R. Heun, O. Benkert and D. F. Levinson, Continuity and discontinuity of affective disorders and schizophrenia. Results of a controlled family study, Arch. Gen. Psychiatry 50 (1993) 871-83.

25. P. Mitchell and D. Tollervey, mRNA turnover, Curr. Opin. Cell Biol. 13 (2001) 320-5.

26. E. Mohr, Subcellular RNA compartmentalization, Prog. Neurobiol. 57 (1999) 507-25.

27. T. Nagase, K. Ishikawa, M. Suyama, R. Kikuno, M. Hirosawa, N. Miyajima, A. Tanaka, H. Kotani, N. Nomura and O. Ohara, Prediction of the coding sequences of unidentified human genes. XII. The complete sequences of 100 new cDNA clones from brain which code for large proteins in vitro, DNA Res. 5 (1998) 355-64.

28. G. Pesole, S. Liuni, G. Grillo, F. Licciulli, F. Mignone, C. Gissi and C. Saccone, UTRdb and UTRsite: specialized databases of sequences and functional elements of 5′ and 3′ untranslated regions of eukaryotic mRNAs. Update 2002, Nucleic Acids Res. 30 (2002) 335-40.

29. R. Prinjha, S. E. Moore, M. Vinson, S. Blake, R. Morrow, G. Christie, D. Michalovich, D. L. Simmons and F. S. Walsh, Inhibitor of neurite outgrowth in humans, Nature 403 (2000) 383-4.

30. S. H. Shaw, M. Kelly, A. B. Smith, G. Shields, P. J. Hopkins, J. Loftus, S. H. Laval, A. Vita, M. De Hert, L. R. Cardon, T. J. Crow, R. Sherrington and L. E. DeLisi, A genome-wide search for schizophrenia susceptibility genes, Am. J. Med. Genet. 81 (1998) 364-76.

31. S. D. Skaper, S. E. Moore and F. S. Walsh, Cell signalling cascades regulating neuronal growth-promoting and inhibitory cues, Prog. Neurobiol. 65 (2001) 593-608.

32. E. F. Torrey, M. Webster, M. Knable, N. Johnston and R. H. Yolken, The Stanley Foundation Brain Collection and Neuropathology Consortium, Schizophr. Res. 44 (2000) 151-5.

33. E. F. Torrey and R. H. Yolken, Familial and genetic mechanisms in schizophrenia, Brain Res. Brain Res. Rev. 31 (2000) 113-7.

34. D. M. Waterwort, A. S. Bassett and L. M. Brzustowicz, Recent advances in the genetics of schizophrenia, Cell. Mol. Life Sci. 59 (2002) 331-48.

35. J. Yang, L. Yu, A. D. Bi and S. Y. Zhao, Assignment of the human reticulon 4 gene (RTN4) to chromosome 2p14→2p13 by radiation hybrid mapping, Cytogenet. Cell. Genet. 88 (2000) 101-2.

36. M. Zagrebelsky, A. Buffo, A. Skerra, M. E. Schwab, P. Strata and F. Rossi, Retrograde regulation of growth-associated gene expression in adult rat Purkinje cells by myelin-associated neurite growth inhibitory proteins, J. Neurosci. 18 (1998) 7912-29.

We claim:

1. A method of determining the susceptibility of a patient to a neuropsychiatric disorder comprising:
(a) obtaining a sample from a patient; and
(b) testing the sample for the presence of a polymorphism in the Nogo gene, wherein the presence of a polymorphism indicates that the patient is susceptible to a neuropsychiatric disorder.

2. A method according to claim 1 wherein the polymorphism is a CAA insert at nucleotides 4386-4388 of the Nogo sequence as shown in FIG. 1 (SEQ ID NO:1).

3. A method according to claim 2 wherein the patient is homozygous for the CAA insert.

4. A method according to claim 3 wherein the neuropsychiatric disorder is schizophrenia.

5. A method according to claim 4 wherein the patient also has a deletion of nucleotides TATC at nucleotide 3905 of the Nogo sequence shown in FIG. 1 (SEQ ID NO:1).

6. The method according to claim 1, wherein the sample is blood.

7. A method according to claim 1 wherein step (b) comprises (i) extracting nucleic acids comprising the Nogo gene, or a portion thereof, from the sample; (ii) amplifying the extracted nucleic acids comprising the Nogo gene, or a portion thereof using polymerase chain reaction (PCR); (iii) performing electrophoresis of the PCR products; and (iv) determining the presence of a polymorphism in Nogo gene.

8. A method according to claim 1 wherein step (b) comprises:
(i) extracting nucleic acids comprising the Nogo gene, or portion thereof, from the sample;
(ii) sequencing the nucleic acids comprising the Nogo gene, or portion thereof; and
(iii) determining the presence of a polymorphism in the Nogo gene.

9. A method of diagnosing a neuropsychiatric disorder in a patient by analyzing for a presence of a polymorphism in the Nogo gene in a biological sample obtained from the patient, wherein the presence of a polymorphism in the Nogo gene, indicates a likelihood that the patient is suffering from a neuropsychiatric disorder.

10. A method according to claim 9 wherein the polymorphism is a CAA insert at nucleotides 4386-4388 of the Nogo sequence as shown in FIG. 1 (SEQ ID NO:1).

11. A method of determining if a patient will have increased symptomatology associated with a neuropsychiatric disorder by analyzing for a presence of a polymorphism in the Nogo gene in a biological sample obtained from the patient, wherein the presence of a polymorphism in the Nogo gene, indicates a likelihood that the patient will have increased symptomatology associated with a neuropsychiatric disorder.

12. A method according to claim 11 wherein the polymorphism is a CAA insert at nucleotides 4386-4388 of the Nogo sequence as shown in FIG. 1 (SEQ ID NO:1).

13. A kit for determining the susceptibility of a patient to a neuropsychiatric disorder comprising (i) reagents for conducting a method according to claim 1 and (ii) instructions for its use.

14. A kit according to claim 13, wherein said reagents comprise nucleic acid primers for amplifying nucleic acids comprising the Nogo gene, or a portion thereof, in a polymerase chain reaction.

15. A kit according to claim 14, wherein the reagents comprise nucleic acid primers for amplifying nucleic acids comprising the Nogo gene, or a portion thereof, in a polymerase chain reaction, DNA polymerase, dATP, dTTP, dCTP, dGTP and buffers.

16. A kit for diagnosing a neuropsychiatric disorder in a patient comprising (i) reagents for conducting a method according to claim 9 and (ii) instructions for its use.

17. A kit for determining if a patient will have increased symptomatology associated with a neuropsychiatric disorder comprising (i) reagents for conducting a method according to claim 11 and (ii) instructions for its use.

18. A kit according to claim 16, wherein said reagents comprise nucleic acid primers for amplifying nucleic acids comprising the Nogo gene, or portion thereof, in a polymerase chain reaction.

19. A kit according to claim 17, wherein said reagents comprise nucleic acid primers for amplifying nucleic acids comprising the Nogo gene, or portion thereof, in a polymerase chain reaction.


SEQUENCE LISTING (6 sequences)

SEQ ID NO: 1 - DNA, 972 bp, Homo sapiens
aaagatgctatggctaaaatccaagcaaaaatccctggattgaagcgcaaagctgaatgaaaacgcccaaaataattagtaggagttcatctttaaaggggatattcatttgattatacggatctttatttttagccatgcactgttgtgaggaaaaattacctgtcttgactgccatgtgttcatcatcttaagtattgtaagctgctatgtatggatttaaaccgtaatcatatctttttcctatctgaggcactggtggaataaaaaacctgtatattttactttgttgcagatagtcttgccgcatcttggcaagttgcagagatggtggagctagaaaaaaaaaaaaaaaagcccttttcagtttgtgcactgtgtatggtccgtgtagattgatgcagattttctgaaatgaaatgtttgtttagacgagatcataccggtaaagcaggaatgacaaagcttgcttttctggtatgttctaggtgtattgtgacttttactgttatattaattgccaatataagtaaatatagattatatatgtatagtgtttcacaaagcttagacctttaccttccagccaccccacagtgcttgatatttcagagtcagtcattggttatacatgtgtagttccaaagcacataagctagaagaagaaatatttctaggagcactaccatctgttttcaacatgaaatgccacacacatagaactccaacaacatcaatttcattgcacagactgactgtagttaattttgtcacagaatctatggactgaatctaatgcttccaaaaatgttgtttgtttgcaaatatcaaacattgttatgcaagaaattattaattacaaaatgaagatttataccattgtggtttaagctgtactgaactaaatctgtggaatgcattgtgaactgtaaaagcaaagtatcaataaagcttatagacttaaaaaaaaaa

SEQ ID NO: 2 - DNA, 38 bp, Artificial Sequence
  primers
tacagcttaaaccacaatggtagcattagattcagtcc

SEQ ID NO: 3 - DNA, 20 bp, Artificial Sequence
  primer
gcatcaggcacagatagatc

SEQ ID NO: 4 - DNA, 22 bp, Artificial Sequence
  primer
tacagcttaaaccacaatggta

SEQ ID NO: 5 - DNA, 18 bp, Artificial Sequence
  primer
ttacctgtcttgactgcc

SEQ ID NO: 6 - DNA, 20 bp, Artificial Sequence
  primer
tacagcttaaaccacaatgg